(19) United States
(12) Patent Application Publication
(10) Pub. No.: US 2009/0311208 A1
(43) Pub. Date: Dec. 17, 2009

(54) Oil In Silicone Emulsions and Compositions Containing Same

(75) Inventors: Mark S. Garrison, Suffern, NY (US); Michael Armstrong, East Rutherford, NJ (US)

Correspondence Address: AVON PRODUCTS, INC., AVON PLACE, SUFFERN, NY, 10901, US

(73) Assignee: Avon Products, Inc., Suffern, NY (US)

(21) Appl. No.: 12/544,475

(22) Filed: Aug. 20, 2009

Related U.S. Application Data
(63) Continuation of application No. 10/747,320, filed on Dec. 26, 2003.

(51) Int. Cl. A61K 8/30; A61K 47/30; A61Q 17/04

(52) U.S. Cl. 424/59; 514/772.3

(57) ABSTRACT

The present invention provides an oil in silicone emulsion and an oil in silicone/water in silicone dual emulsion. These emulsions employ a silicone resin as the emulsifying agent, include an effective amount of one or more active ingredients, such as, a sunscreen and/or insect repellent and provide enhanced aesthetics and SPF. Also provided are methods of preparation of the oil in silicone and the oil in silicone/water in silicone dual emulsions, compositions that employ the emulsions and methods of using the compositions to repel insects from skin and protect skin from exposure to the sun.

### CROSS REFERENCE TO PRIOR APPLICATION

This application is a continuation of U.S. Ser. No. 10/747,320, filed Dec. 26, 2003, the disclosure of which is incorporated by reference.

### BACKGROUND OF THE INVENTION

1. Field of the Invention

The present invention relates to an oil in silicone emulsion, cosmetic compositions thereof and methods of their use in a variety of sun care, skin care, personal care and cosmetic products to provide increased sun protection benefits with reduced level of sunscreen required for effective sun protection. More particularly, the present invention relates to an oil in silicone/water in silicone dual emulsion, cosmetic compositions thereof and methods of their use.

2. Description of the Prior Art

Increased stability, improved aesthetics and increased sun protection benefits with reduced level of sunscreen required for effective sun protection and other performance characteristics are desirable attributes in sun care, skin care, hair care, personal care, and cosmetic products, compositions and preparations.

For example, water resistance is a much-desired quality amongst consumers of color cosmetics, lipsticks, eyelash mascara, insect repellents and sunscreen preparations. Additionally, shine and gloss are much desired attributes in nail color and hair care preparations. Thus, there is a need in consumer products and cosmetic industry for formulations that can enhance the effectiveness of the various active ingredients contained therein and can deliver multiple benefits, such as, increased stability, increased performance characteristics, and increased sun protection benefits with reduced level of sunscreen required for effective sun protection.

U.S. Pat. No. 6,262,170 describes a composition containing a swollen gel network of a silicone elastomer of MDTQ structural type. Such elastomers are generally insoluble. The elastomer is swollen by a liquid and thereafter subjected to a high shearing force to break the elastomer into particles.

In contrast to this approach, the current invention utilizes a silicone resin, which is soluble in silicone fluid instead of being merely swellable. The silicone resins of the present invention are of the chemical type MDTQ, preferably of the MQ type, which are not elastomers. When dissolved, the silicone resins utilized in the present invention exhibit rheological properties that are different from the rheological properties of elastomers. They are not swollen matrices and do not require high shearing forces to break them into particles.

U.S. Pat. No. 6,238,657 uses elastomers, not silicone resins, to form oil in silicone emulsions. The oils are natural oils, such as vegetable oils. There is no mention of any composition that has an enhanced SPF or any other performance characteristic.

U.S. Pat. No. 6,346,553 describes the use of alkylmethylsiloxane-dimethylsiloxane-polyalkylene oxide copolymers as emulsifiers. However, this patent does not mention silicone resins, which belong to an entirely different class of compounds. It does not teach or disclose the use of silicone resins as emulsifiers for oil-in silicone emulsions, nor does it teach an enhanced SPF. The SPF enhancing effect of oil in silicone sunscreen compositions according to the present invention is not described in this art or in any prior art.

U.S. Pat. No. 6,517,816 describes a metastable sunscreen water-in-oil emulsion composition having an inner discontinuous phase and an outer continuous phase. The inner discontinuous phase and/or outer continuous phase has a sunscreen active therein. The inner discontinuous phase is generally dispersed in the outer continuous phase and is in the form of discrete droplets having a multimodal droplet size distribution. It does not describe oil in silicone emulsions or oil in silicone/water in silicone dual emulsions that are stable rather than metastable.

None of the art described above mentions enhanced SPF from oil in silicone emulsions or the use of silicone resins as the oil in silicone emulsifiers. In fact, sunscreen compositions that are oil in silicone emulsions are not even mentioned in the art described above. Further, the art described above does not teach the use of the present combination of ingredients to achieve high SPF sunscreen compositions by utilizing MDTQ silicone resins, especially MQ resins, to emulsify the oil phase into the silicone fluid.

### SUMMARY OF THE INVENTION

It is an object of the present invention to provide oil in silicone emulsions useful in the formulation of cosmetic compositions, especially sunscreen compositions.

It is a further object of the present invention to provide oil in silicone/water in silicone dual emulsions useful in the formulation of cosmetic compositions, especially sunscreen compositions.

It is still a further object of the present invention to provide compositions having the oil in silicone emulsion or the oil in silicone/water in silicone dual emulsion of the present invention.

It is another object of the present invention to provide a product, such as, sun care, skin care, personal care and other cosmetic products having a sunscreen as an active ingredient.

It is yet another object of the present invention to provide an enhanced SPF product having a sunscreen as an active.

It is another object of the present invention to provide an enhanced SPF product with the dual emulsion of the present invention, which has superior aesthetics, superior lubricity and a non-greasy feel.

It is still another object of the present invention to provide a composition having an insect repellent as an active ingredient.

It is yet another object of the present invention to provide methods of preparing these emulsions and products.

The present invention provides such oil in silicone emulsions and such oil in silicone/water in silicone dual emulsions.

The oil in silicone emulsion includes an internal oil phase having an effective amount of a lipophilic and/or hydrophobic active ingredient, a silicone external phase having a silicone fluid, and a silicone resin in an amount sufficient to emulsify the oil into the silicone fluid to produce the oil in silicone emulsion.

The oil in silicone/water in silicone dual emulsion includes an internal oil phase having an effective amount of an active ingredient, an internal aqueous phase, a silicone external phase having a silicone fluid, a silicone resin in an amount sufficient to emulsify the oil into the silicone fluid to produce the oil in silicone emulsion within the dual emulsion, and an emulsifier for stabilizing the water droplets within the dual emulsion.

The present invention further provides a cosmetic composition, which has the oil in silicone emulsion or the oil in silicone/water in silicone dual emulsion according to the present invention and one or more additional ingredients constituting a cosmetically or pharmaceutically acceptable vehicle.

The present invention still further provides a process for preparation of an oil in silicone emulsion, including the steps of: mixing an effective amount of one or more lipohilic and hydrophobic active ingredients and optionally an oil and/or an adjuvant at sufficient shear at a temperature and for a period of time sufficient to produce an oil phase; mixing a silicone fluid and a silicone resin and optionally an adjuvant at sufficient shear at a temperature and for a period of time sufficient to produce a silicone phase; and adding the oil phase to the silicone phase at sufficient shear at a temperature and for a period of time sufficient to produce the oil in silicone emulsion.

The present invention also provides a process for preparation of an oil in silicone/water in silicone dual emulsion, which includes the steps of: mixing an effective amount of one or more lipohilic and hydrophobic active ingredients and optionally an oil and/or an adjuvant at high shear at a temperature and for a period of time sufficient to produce an oil phase; mixing a silicone fluid and a silicone resin and optionally an adjuvant at high shear at a temperature and for a period of time sufficient to produce a silicone phase; adding the oil phase to the silicone phase at high shear at a temperature and for a period of time sufficient to produce an oil in silicone emulsion; mixing water and one or more water-soluble ingredient to form a water phase; and adding the water phase to the oil in silicone emulsion at high shear at a temperature and for a period of time sufficient to produce the oil in silicone/water in silicone dual emulsion.

The above emulsions and compositions having a sunscreen active provide higher than expected SPFs. Thus, if water is added into the oil in silicone emulsion to form an additional internal phase, it is unexpectedly found that the enhancement in SPF performance obtained by the oil in silicone emulsion is not adversely affected and, as a result, a higher SPF is maintained with a given amount of sunscreen. Conversely, lower amount of sunscreen provides the given SPF.

The advantages of the sunscreen-containing oil in silicone emulsions and the sunscreen-containing oil in silicone/water in silicone dual emulsions according to the present invention include increased sun protection with reduced level of sunscreen required. Reducing the amount of sunscreen required to achieve a given SPF results in a more efficient, cost effective, and less irritating product.

A further significant advantage of the emulsions described herein is the improved aesthetics of high SPF products over conventional emulsions. The utilization of the silicone resin at the levels described in the oil in silicone or dual emulsions described provides a unique non-greasy, light, fast drying, non-sticky, aesthetically pleasing product. Such products are unique in high SPF waterproof sunscreens.

Through the combination of the silicone resin and the silicone external phase, the present compositions are inherently waterproof, without the need for PVP derived or other film formers.

The compositions of the present invention have emulsion stability and provide parity active agent benefits at a reduced level of active agent or increased active agent benefit at parity active agent concentration.

The present compositions provide good waterproof performance and superior aesthetics in sunscreen and other cosmetic and personal care products applications. Unexpectedly, when the resin is used to form these compositions, the combination of benefits described herein is achieved.

These and other objects and advantages of the present invention are achieved by the use of a silicone resin to form an oil in silicone emulsion or an oil in silicone/water in silicone dual emulsion.

### DETAILED DESCRIPTION OF THE INVENTION

The present invention provides an oil in silicone emulsion, which can be used in a variety of sun care, skin care, hair care and personal care and other cosmetic products.

Compositions containing a sunscreen agent are preferred embodiments of the present invention.

The oil in silicone emulsion according to the present invention is particularly useful in personal care products that provide SPF protection, especially products that provide high SPF protection, typically 30 and above, including sunscreen containing insect repellent products.

The oil in silicone emulsion according to the present invention is also useful in personal care products that require water resistant performance.

The unique oil in silicone emulsion according to the present invention provides stable sunscreen product compositions of given sun protection factor (SPF) benefit at a reduced level of sunscreen concentration, or increased SPF benefit at a given sunscreen active concentration. In addition, the oil in silicone emulsion according to the present invention provides good waterproof performance and exceptional aesthetics. The emulsion has a silky feel, with a very rapid dry down, and a very light and pleasant feeling. Such properties are highly desirable in high SPF product, i.e., products that have an SPF above 30.

An essential aspect of the present invention is the formation of an oil in silicone emulsion. Such oil in silicone emulsions are very different from the traditional emulsions, which are either oil in water or water in oil emulsions.

It has been unexpectedly found that the MDTQ silicone resins, especially the MQ silicone resins, are capable of emulsifying an array of lipophilic and/or hydrophobic compounds with diverse structures. The silicone resins function as emulsifiers/emulsion stabilizers when combined with silicone fluids, for example, dimethicone, and are able to emulsify immiscible hydrophobic oils into the silicone fluid. The silicone resins used to form the oil in silicone emulsion are referred to herein as silicone emulsifying resins.

The emulsions are stable when made and such stability extends through the manufacture of the compositions of the present invention. The compositions of the present invention, which can include suitably stable emulsions according to the present invention, are stable for at least about one month at 25° C., preferably for one month at 40° C., and most preferably for two months at 40° C.

A preferred class of compounds that can form the oil phase of the oil in silicone emulsion includes cyclic compounds having two or more carbon rings and especially dicyclic compounds. The carbon rings can be cycloaliphatic, aromatic, fused, or combinations thereof. Thus, the liquid lipophilic and/or hydrophobic compounds, such as, homomethyl salicylate, and menthyl anthranilate, could form such emulsions.

The solid lipophilic and/or hydrophobic dicyclic compounds, such as, methyl benzylidene camphor, benzophenone-4, and butylmethoxydibenzoylmethane, would form the oil in silicone emulsions if dissolved.

Another preferred class of compounds that can form the oil phase in the oil in silicone emulsions disclosed herein are hydrophobic and lipohilic compounds containing one or more branched long chain aliphatic hydrocarbons of C8 to C24 carbons, especially C8 to C18 carbons, having at least one branched hydrocarbon chain, such as certain esters with C8 to C24 branched alkyl chains, especially aliphatic chains. Ethyl hexyl, iso-octyl, isocetyl, isopalmityl, isostearyl and isostearate chains are especially preferred.

The preferred oils include trimethylolpropane tri-isostearate, ethylhexyl salicylate, triethylhexyl citrate, di-isostearyl fumarate and ethyl hexyl hydroxyl stearate.

The ability of the resin to emulsify an oil into dimethicone decreases as the number of carbons in the chain decreases. Thus, the C16 or C18 isohydrocarbon chain is more stable when emulsified as compared to the C8 (ethyl hexyl) moiety. Linear alkyl chains, i.e., those from natural fatty acids such as stearate, palmitate, myristate, etc., are found to be less preferred, as they form less stable emulsions.

It is important to note that the compound octocrylene is dicyclic and possesses an ethyl hexyl moiety. It is found to form particularly stable emulsions, sometimes spontaneously forming a paste or gelled emulsion.

The compositions based on these emulsions show superior non-greasy aesthetics, in spite of having a high oil content of normally cosmetically unaesthetic oil, such as, octocrylene.

Because the oil is in the internal phase of the composition, its feel is hidden from the skin. Because the external phase is silicone, the external phase is felt on the skin and, as a result, the composition feels particularly silky. In addition, because the external phase contains a significant proportion of a nonvolatile silicone, the aesthetic is particularly pleasant.

When used at the concentration herein, the silicone resin serve an additional function of giving the formula a dry after-feel in addition to making the stable emulsion possible. This dry after-feel is further augmented if the formula further contains inorganic powders.

A highly desirable property of these emulsions is that the compositions exhibit unexpected enhanced SPF properties when formulated as sunscreen products.

To obtain an oil in silicone emulsion, the emulsification must be carried out in the unique manner in accordance with the present invention. It is most surprising that such emulsions exhibit an enhanced SPF over the traditional emulsions known in the art.

The formation of an oil in silicone emulsion using silicone resin as the emulsifier is surprising, and the enhanced SPF provided by such an emulsion is even more surprising.

### Active Ingredient

The lipophilic and/or hydrophobic active ingredient can be used as the oil phase, without an additional oil being present in the emulsion. In such cases, the active ingredient is emulsifiable in the silicone phase such that the active ingredient alone becomes the oil phase. This can be the case in a high SPF product, where the oil phase is primarily the sunscreen active ingredients.

In cases where the lipophilic and/or hydrophobic active ingredient is used in combination with an oil, it is preferred that the active ingredient is soluble in the oil and that the combination is emulsifiable in the silicone phase.

The active ingredients that can be used are generally lipophilic and/or hydrophobic. Such lipophilic and/or hydrophobic active ingredients include sunscreens, insect repellent, anesthetic, anti-allergenic, antifungal, antimicrobial, anti-inflammatory agent, antiseptic, exfollient, pharmaceutical, and any combinations thereof. Preferably the active agent is both lipophilic and hydrophobic.

Preferably, the active ingredient has an octanol-water partition coefficient whose log, referred to herein as Log P, is at least 3.5, more preferably at least 4.5, and most preferably 5.0 or greater. The octanol-water partition coefficient parameters described in Kirk-Othmer Encyclopedia of Chemical Technology, Fourth Edition, Volume 9, John Wiley & Sons, New York, N.Y., page 478 (1994), and in respect of the present invention, was calculated with software available from the U.S. Environmental Protection Agency, EPI Suite v. 3.11, available at www.epa.gov/oppt/exposure/docs/episuitedl.htm.

Preferably, the active ingredient is a sunscreen active, an insect repellent or any mixtures thereof. If the active is a sunscreen, it is preferable that it contain two or more hydrocarbon rings for maximum emulsification or that it contain a branched alkyl chains of C8 or higher.

Examples of such lipophilic and/or hydrophobic sunscreen actives include oxybenzone, dioxybenzone, menthyl anthranilate, benzophone-3, octyl methoxycinnamate, octocrylene, drometrizole trisiloxane, octyl salicylate, homomethyl salicylate, 4-methyl benzylidene camphor, 3-benzylidene camphor, benzylidene camphor sulfonic acid ester, octyl triazone, phenyl benzimidazole sulfonic acid ester, terephthalydiene dicamphor sulfonic acid ester, di-t-butyl hydroxybenzylidene camphor, dibenzoylmethane derivatives, such as, butylmethoxy dibenzoylmethane (avobenzone), (Parsol 1789), terephthalydiene methylene bis-benzotriazolyl-tetramethylbutylphenol, diethylhexyl-2,6-naphthalate, bis-ethylhexyloxyphenol methoxyphenol triazine, benzotriazole derivatives, such as, hydroxy methylphenyl benzotriazole, methylene bis-benzotriazolyltetramethylbutyl phenol, bis-ethylhexyloxyphenol methoxyphenol triazine, hydroxybenzophenone, an oxanilide, cinnamate derivatives, such as ethylhexyl methoxy cinnamate, isocetyl salicylate, a silicone-anchored sunscreen, such as, polysilicone-12, and any combinations thereof. Oil dispersible particulate sunscreen agents such as zinc oxide and titanium dioxide, which are made dispersible, for example by treatment with stearic acid or octyl silane, are suitable for use when admixed with a lipophilic and/or hydrophobic oil as described herein and thereafter used to form the oil in silicone emulsion.

The active ingredient is a physiologically acceptable compound. In the context of the present invention, the term “physiologically acceptable” refers to any ingredient, such as, a drug, a cosmetic, a medicament or an optional ingredient that is suitable for use in direct, safe contact with human tissues.

Actives that are not physiologically acceptable are excluded from this invention. PABA and derivatives of PABA are known to cause contact dermatitis and/or photo contact dermatitis. Therefore, PABA and derivatives of PABA are excluded from this invention.

Examples of such actives that are physiologically not acceptable include para aminobenzoic acid (PABA), para aminobenzoic acid esters, para aminobenzoic acid amides, para-N,N-dimethylaminobenzoic acid esters and other derivatives of PABA.

Preferably, the sunscreen composition includes at least two sunscreen actives, and more preferably three sunscreen actives.

The effective amount of the sunscreen active for providing sun protection benefit is about 1 wt % to about 50 wt % of the total weight of the emulsion, preferably, about 1.0 wt % to about 40 wt % of the total weight of the emulsion, most preferably, about 10 wt % to about 35 wt % of the total weight of the emulsion.

Preferably, the emulsion exhibits an SPF about 2 to about 100. More preferably, the emulsion exhibits an SPF about 30 to about 100, especially about 30 to about 75, and most preferably, the emulsion exhibits an SPF about 50 to about 65.

Hydrophilic sunscreen actives, i.e., those that are water soluble or water dispersible, such as, TEA salicylate, benzylidene camphor sulfonic acid, phenyl benzimidazole sulfonic acid, sulisobenzone, terephthalydiene dicamphor sulfonic acid, hydroxy cinnamic acid, and combinations thereof can be incorporated into the internal aqueous phase of an oil in silicone/water in silicone dual emulsion according to the present invention.

In such emulsions, the hydrophilic sunscreen active resides within the water droplets that forms a separate internal phase of the oil in silicone/water in silicone dual emulsion. As known in the art, the hydrophilic sunscreens are typically neutralized to form water soluble salts prior to use.

Thus, the emulsions according to the present invention can simultaneously have one or more lipophilic and/or hydrophobic sunscreen active in the oil phase and one or more hydrophilic sunscreen active in the aqueous phase.

In the case where the active ingredient is a mixture of a sunscreen and an insect repellent, the mixture of the sunscreen and insect repellent is up to about 70 wt % of the total weight of the composition, preferably, about 1.0 wt % to about 50 wt % of the total weight of the emulsion, most preferably, about 10 wt % to about 40 wt % of the total weight of the emulsion.

Typically, the lipophilic active would be in the oil phase of the oil in silicone emulsion, and the hydrophilic actives, if any, would be provided in the water phase of the dual emulsion system.

The preferred insect repellents include oil of citronella, DEET, and IR 3535, which has the following chemical formula:

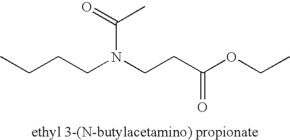

Ethyl 3-(N-butylacetamino) propionate is sold under the commercial name Merck IR 3535 by Merck Corporation, and can be added to the water phase of the oil in silicone/water in silicone dual emulsion, as it is somewhat hydrophilic.

The effective amount of the insect repellent active for providing insect protection benefit is about 0.1 wt % to about 50 wt % of the total weight of the emulsion. More preferably, the effective amount of the insect repellent active is about 2.5 wt % to about 35 wt % of the total weight of the emulsion and most preferably, the effective amount of the insect repellent active is about 5 wt % to about 20 wt % of the total weight of the emulsion.

### Oil

The oils used to form the oil in silicone emulsion of the present invention are characterized as being immiscible with the silicone fluid in the absence of the silicone emulsifying resin, that is, forming two separate phases when mixed in the absence of the resin—an oil phase and a silicone fluid phase.

Preferably, the oil is a branched mono-, di-, tri- or tetra-ester of a polyfunctional alcohol having a functionality of up to four with a fatty acid of 8 to 50 carbon atoms, preferably up to about 22 carbon atoms and any combinations of any of the polyfunctional alcohols and any of the 8 to 50 carbon fatty acids.

Examples of such esters include a triester of trimethylol-propane, di-trimethylolpropane tetra-isostearate, an alkoxylated derivative thereof, diisostearyl fumarate, and any mixtures thereof. Preferably, the ester is di-trimethylolpropane-tetra-isostearate.

Also useful are aromatic esters, that is esters where either the acid or alcohol is aromatic. Especially useful is the 2-phenylethyl ester of benzoic acid. This ester has two aromatic groups. It is available commercially under the trade name X-TEND 226 and is marketed by ISP technologies.

The oil can be the active itself without any additional oil being present. Preferably, such an active is both lipophilic and hydrophobic.

Preferably, the oil is present up to about 50 wt % based on the total weight of the emulsion. More preferably, the oil is present at about 1 wt % to about 35 wt % based on the total weight of the emulsion and most preferably, the oil is present at about 5 wt % to about 30 wt % based on the total weight of the emulsion.

### Silicone Fluid

Background material on silicones including sections discussing silicone fluids and resins are found in Encyclopedia of Polymer Science and Engineering, Volume 15, Second Edition, pages 204-308, John Wiley & Sons, Inc., 1989, and Chemistry and Technology of Silicones, Walter Noll, Academic Press, Inc. (Harcourt Bruce Javanovich, Publishers, New York), 1968, pages 282-287 and 409-426, both incorporated herein by reference.

The silicone fluid used to make the emulsions of the present invention is preferably a nonvolatile silicone fluid. The silicone fluid in the oil in silicone emulsions according to the present invention can optionally include a volatile silicone fluid in limited amounts, as hereinafter defined.

Suitable nonvolatile silicone fluids have from 1 to 100 siloxy units and generally have a viscosity of about 25,000 cS or less, preferably about 5,000 cS or less, more preferably about 1,000 cS or less, and most preferably less than about 100 cS, at 25° C. Viscosity will generally be at least about 5 cS, more preferably about 10 cS, at 25° C.

Suitable silicone fluids include polyalkyl siloxanes, polyaryl siloxanes, polyalkylaryl siloxanes, and any mixtures thereof. More particularly, silicone fluids include polysiloxanes having the structure:

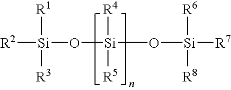

wherein R1, R2, R3, R4, R5, R6, R7 and R8 can independently be aliphatic, preferably alkyl, aryl, alkoxy, aryloxy, alkaryl, and arylalkyl. These R groups can be further substituted, e.g., with a halogen. The integer x has a value of from 1 to 100, preferably from 5 to 50, especially from 10 to 25.

The aliphatic or aryl groups substituted on the siloxane may have any structure so long as the resulting silicone is hydrophobic and not miscible with the oil component or the lipophilic and/or hydrophobic active ingredient. Preferred aliphatic chains for the radicals R1-R8 are C1 to C6 alkyls, preferably C1 to C4 alkyls, and most preferably methyl or ethyl.

These siloxanes are available, for example, from the General Electric Company in their Viscasil R and SF 96 series, and from Dow Corning in their Dow Corning 200 series.

Other suitable non-volatile silicone fluids include polydimethyl siloxane (dimethicone), polydiethylsiloxane, and polymethylphenylsiloxane. Polydimethylsiloxane is especially preferred.

Additional suitable non-volatile silicone fluids are described in greater detail in International Application WO 95/05800, the contents of which are incorporated herein by reference as fully set forth.

The viscosity of the volatile silicone fluids suitable for use in the present emulsions is generally about 100 cS (centislokes) or less at 25° C., preferably about 50 cS or less, especially less than about 10 cS, all determined at 25° C.

As used herein, the term “volatile” refers to liquids having a boiling point at one atmosphere of 260° C. or less, preferably 250° C., more preferably 230° C. or less, most preferably 225° C. or less.

In general, the boiling point of the volatile solvents will be at least about 50° C., preferably at least about 100° C. The term “nonvolatile” on the other hand, refers to materials that have a boiling point greater than 260° C. at one atmosphere.

Volatile silicone fluids suitable for use herein include both linear and cyclic silicone fluids. Examples of such volatile silicone fluids include trisiloxane, volatile dimethicones, methyl trimethicone, and any mixtures thereof.

Examples of the cyclic volatile silicone fluids include cyclopolysiloxanes, such as, cycloalkylsiloxanes and cycloalkyalkoxysiloxanes, wherein alkyl and alkoxy groups contain C₁-C₈ alkyl groups. Specific examples include octamethyl cyclotetrasiloxane (cyclomethicone tetramer), decamethyl cyclopentasiloxane (cyclomethicone pentamer), cyclomethicone hexamer, and methoxypropyl heptamethyl cyclotetrasiloxane.

Volatile silicone fluids can be used in the emulsions of the present invention but only in limited amounts. Examples of linear polyorganosiloxanes include those having from about 3 to about 9 silicon atoms and can have saturated or unsaturated C₁-C₈ alkyl, aryl, (preferably containing a 6 carbon aromatic ring), hydroxyalkyl, amino alkyl or alkyl siloxy with up to 7 repeating units.

The preferred volatile linear polydimethylsiloxanes have from about 3 to 9 silicone atoms and are polydialkylsiloxanes, especially those with C₁-C₂, preferably C1 alkyls. Examples of preferred linear materials include polydialkylsiloxanes, such as, polydimethylsiloxane having viscosity below about 10 CS at 25° C., and trisiloxane.

The trisiloxane can be used either alone or can be blended with volatile dimethicone, such as, for example, Dow Corning 2-1184 fluid, which is commercially available from Dow Corning.

The linear volatile silicones generally have viscosities of 5 cS (centistokes) or less at 25° C., while the volatile cyclic materials generally have viscosities of 10 cS or less at 25° C. Volatile silicones are described in Todd and Byers, “Volatile Silicone Fluids for Cosmetics,” Cosmetics and Toiletries, Vol. 91, January, pages 27-32 (1976) the contents of which are incorporated herein by reference.

The nonvolatile polyalkylsiloxane fluids that can be used in the present invention include, for example, polydimethyl-siloxanes.

Polyalkylaryl siloxane fluids can also be used, but only in small quantities because they solubilize the sunscreen actives and “break” the emulsion. Examples of polyalkylaryl fluids include polymethyl-phenylsiloxanes, available, for example, from the General Electric Company as SF 1075 methyl phenyl fluid or from Dow Corning as 556 Cosmetic Grade Fluid.

Alkyl polysiloxanes, which are silicone waxes, can be used in small amounts to add lubricity. These include C₁₂C₂₂ alkyl methyl polysiloxanes, such as, cetyl and stearyl dimethicone, and behenoxy dimethicone. Such examples include Abil-Wax 9800™, a stearyl dimethicone available from Goldschmidt, and Abil-Wax 9801™, a cetyl dimethicone, and Abil Wax 2440™ a behenoxy dimethicone.

The amount of the silicone fluid can be up to about 70 wt % of the total weight of the emulsion. Preferably, the silicone fluid is present at about 10 wt % to about 50 wt % of the total weight of the emulsion.

Preferably, the silicone fluid is a non-volatile silicone fluid or it is a mixture of a non-volatile silicone fluid and a volatile silicone fluid. Thus, some volatile silicone fluid can be present in the silicone fluid. However, the amount of the volatile silicone fluid should be kept at a minimum amount necessary without destroying the emulsion. Therefore, the volatile silicone fluid must not exceed the amount that destroys the emulsion.

Typically, when the total of silicone resin and nonvolatile silicone is present at about 20%, the amount of volatile silicone should be less than about 20%, i.e., should not exceed the amount of the combined nonvolatile and resin silicone components.

The volatile silicones can add a pleasant feeling to the composition, but if it is used without a non volatile dimethicone, or if it is incorporated at too high a concentration, the volatile silicone will form an unstable emulsion, which will separate within a few days. This outcome is undesirable and necessitates that the product is shaken vigorously to re-emulsify prior to use.

Thus, the volatile silicone fluid can be up to 50 wt % of the total weight of the silicone fluid/silicone resin mixture provided that the integrity and the stability of the emulsion are maintained. Preferably, the amount of volatile silicone is less than about 15 wt % of the total weight of the composition, and most preferably less than about 5 wt % by weight of the total composition.

The destabilizing effect of the volatile silicones can be somewhat counteracted by the use of silicone treated powders. Examples include silane or silicone treated talc, TiO2, ZnO, or silica. These are typically used at about 0.1 wt % to about 15 wt %. When these powders are silicone treated titanium dioxide or zinc oxide, they serve a dual purpose in that they also are inorganic sunscreens.

The preferred non-volatile silicone fluid is dimethicone with a viscosity about 5 to about 100 cS. The preferred volatile silicone fluid is cyclomethicone (pentamer).

Mixtures of dimethicone and cyclomethicone are also preferred, provided that the amount of the volatile silicone fluid is such that the integrity and the stability of the emulsion are maintained.

### Silicone Emulsifying Resins

Silicone resins can conveniently be identified according to a shorthand nomenclature system well known to those skilled in the art as “MDTQ” nomenclature. Under this system, the silicone is described according to presence of various siloxane monomer units that make up the silicone. Briefly, the symbol M denotes the monofunctional unit (CH₃)₃SiO_{0.5}, D denotes the difunctional unit (CH₃)₂SiO, T denotes the trifunctional unit (CH₃)SiO_{1.5}, and Q denotes the tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc.

The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′M and/or M′ in a silicone resin is indicative of higher levels of crosslinking. However, the overall level of crosslinking can also be indicated by the oxygen to silicone ratio.

The preferred silicone resins that are suitable for use herein are MQ, MT, MTQ, MDQ and MDTQ resins, in which the substituent is methyl. However, resins in which the substituent is partially or completely ethyl are also useful.

Especially preferred are the MQ resins and the M′Q′ resins (where the substituent is ethyl), particularly those wherein the M:Q or M′:Q′ molar ratio is from about 0.5:1.0 to about 1.5:1.0 and the weight average molecular weight of the resin is from about 1000 to about 10,000. The MQ resins are most preferred. Examples of such silicone emulsifying resins include trialkylsiloxysilicates, i.e., trimethylsiloxy-silicate.

Examples of MQ type resins include those listed under the INCI name trimethylsiloxysilicate. Specific examples include Dow Corning 593 Fluid from Dow Corning; GE SR399 available from GE Silicones; KF-7312 and KF-9000 resins from Shin Etsu, and TMS 803 (an ethyl modified resin) from Wacker. The latter resin is also available from ISP. The commercially available resins typically are sold premixed with a silicone fluid, usually a nonvolatile silicone fluid, such as dimethicone.

Preferably, the silicone emulsifying resin is up to 30 wt % of the total weight of the silicone fluid. More preferably, the silicone emulsifying resin is about 2.5 wt % to about 15 wt % of the total weight of the emulsion. Most preferably, the silicone emulsifying resin is about 5 wt % to about 10 wt %.

It is critical how the components are mixed in specific amounts and in a particular order of addition in order to form the proper emulsion. One must use enough silicone and enough resin in order to form the oil in silicone emulsion. Merely adding the silicone and silicone resin to a typical emulsion does not yield an oil in silicone emulsion nor does it provide enhanced SPF.

Additional esters or other ingredients are acceptable in the oil phase, but excessive amounts can destabilize the oil in silicone emulsion. The oils described herein do not destabilize the oil in silicone emulsion.

### Emulsion

The present invention also provides a process for preparation of an oil in silicone emulsion.

The process includes the following steps:

mixing an effective amount of one or more cosmetically active ingredients and optionally an oil and/or an adjuvant at high shear, i.e., homogenization with a Silversen stirrer at approximately 5000 rpm for about 5-15 minutes, at a temperature and for a period of time sufficient to produce an oil phase (in some cases, emulsions have been prepared successfully with considerably less shear);

mixing a silicone fluid and a silicone emulsifying resin and optionally an adjuvant at a temperature and for a period of time sufficient to produce a uniform homogeneous silicone phase; and

adding the oil phase to the silicone phase preferably at high shear at a temperature (generally room) and for a period of time, typically 5 to 15 minutes, sufficient to produce the oil in silicone emulsion.

Details of the process are described in the Examples.

### Dual Emulsion

The present invention also provides an oil in silicone/water in silicone dual emulsion.

The dual emulsion can be prepared by a process, which includes the following steps:

mixing an effective amount of one or more cosmetically active ingredients and optionally an oil and/or an adjuvant at high shear at a temperature and for a period of time sufficient to produce an oil phase;

mixing a silicone fluid and a silicone emulsifying resin and optionally an adjuvant (at high shear if such is necessary to dissolve the resin or to disperse silicone treated powders such as TiO₂) at a temperature and for a period of time sufficient to produce a silicone phase;

adding the oil phase to the silicone phase at high shear at a temperature and for a period of time sufficient to produce an oil in silicone emulsion;

mixing water and one or more water-soluble ingredients to form a water phase; and

adding the water phase to the oil in silicone emulsion at low shear at a temperature and for a period of time sufficient to produce the oil in silicone/water in silicone dual emulsion.

The water phase is added slowly over about 20-40 minutes to the oil in silicone emulsion after the initial emulsion is formed. If added too quickly, it can destroy or destabilize the emulsion.

The emulsion is then allowed to mix for a period of time, about 20 to 40 minutes at low shear. To increase stability and viscosity, the emulsion can be further subjected to high shear if such is desired. If shear is excessive the emulsion can be destabilized. Usually it is best to mill for 5-30 minutes only, depending on the device used. During this time the temperature of the batch should be maintained as close to room temperature as possible, and preferably below 100 degrees, until the milling is complete.

When present, water must be added after the oil in silicone emulsion is formed. Both examples 2 and 3 have an oil in silicone emulsion. If water is incorporated into the oil in silicone emulsion, water must be incorporated as an internal phase. Thus, the placement of the water is critical to avoid disruption of the SPF enhancement of the oil in silicone emulsion.

If it is desired to add water to the oil in silicone emulsion, water in silicone emulsifiers such as dimethicone copolyols, peg silicones, peg/ppg silicones or alkylated derivatives thereof can help to stabilize the water droplets. A particularly effective combination of emulsifiers is dimethicone copolyol and cetyl dimethicone copolyol. Examples of such emulsifiers include, but are not limited to, Dow Corning 5225C (a blend of Cyclomethicone and PEG/PPG-18/18 dimethicone), Abil EM-90 (Cetyl PEG/PPG-10/1 dimethicone), Dow Corning Q2 5200 (Lauryl PEG/PPG-18/18 methicone) and any mixtures thereof.

In addition, the inclusion of a salt, such as, sodium chloride, potassium chloride, sodium bromide, potassium bromide, magnesium chloride, magnesium sulfate, sodium sulfate, potassium sulfate, or any mixtures thereof, can improve stability.

Further details of this process steps and conditions are described in the Examples.

In the emulsions, the oil phase is at up to about 50 wt % of the total weight of the emulsion, and preferably about 1 wt % to about 35 wt % of the total weight of the emulsion; the silicone fluid phase is at up to about 70 wt % of the total weight of the emulsion, and preferably about 10 wt % to about 50 wt % of the total weight of the emulsion; and the silicone emulsifying resin is at up to 30 wt % of the total weight of the emulsion, and preferably from about 2.5% to about 15 wt % of the total weight of the emulsion.

The aqueous phase, if present, is from 0.01 to 50 wt % of the total weight of the emulsion, and preferably from about 10 wt % to about 35 wt % of the total weight of the emulsion.

In a preferred embodiment a particularly effective composition is obtained when the oil, water, and silicone phases are in at approximately 25-30 wt % each, and the composition contains about 10-15 wt % inorganic powder such as silicone treated Titanium dioxide.

### Optional Ingredients

The present compositions typically have one or more optional ingredients, generally adjuvants and/or excipients, in addition to the silicone fluid, the silicone resin, and the oil components of the oil phase, including the lipophilic and/or hydrophobic active agent, all present in the oil in silicone emulsion, and further in addition to the water and emulsifier components of the water phase of the dual emulsion.

Preferably, the optional ingredients are physiologically acceptable or suitable for use in the emulsions of the invention. In the context of the present invention, the term “physiologically acceptable” refers to any ingredient, such as, a drug, a cosmetic, a medicament or an optional ingredient that is suitable for use in direct, safe contact with human tissues.

The optional ingredients should also be compatible with the emulsions of the present invention, that is should not substantial destabilize the emulsions, not substantially interfere or compromise any of the other benefits obtained by the practice of the present invention.

These optional ingredients may be incorporated in either the oil phase, the silicone phase, the water phase, or may be present as yet another phase. The optional ingredients may be present in a phase of the present invention as a discreet phase, or may be in the oil or water phase.

Suitable adjuvants and excipients include antioxidants, buffering agents, botanical extracts, chelating agents, pigments, colorants, self tanning agents, depigmenting agents, emollients, film formers, fragrances, humectants, lubricants, moisturizers, conditioning agents, preservatives, skin protectants, skin penetration enhancers, stabilizers, surfactants, thickeners, viscosity modifiers, vitamins, and any combinations thereof.

These ingredients preferably are added to the phase in which they are most miscible.

As stated above, the emulsion may contain adjuvant placed into the phase they are most miscible with. Thus, the oil phase of the emulsion can have one or more additional organic compounds, including, for example, esters benzyl alcohol or methyl paraben, which function as preservatives. The aqueous phase can have for example humectants, such as propylene glycol, glycerin, or polyethylene glycol. Other water-dispersible or water-soluble components may be included. Aqueous thickeners such as veegum or hydroxyalkyl cellulose, or gelling agents, such as high MW polyacrylic acid, i.e., Carbopol 934, and any mixtures thereof, can be used, but preferably sparingly, as for proper emulsification as an internal phase of the dual emulsion, the viscosity of the water phase should be kept low, less than about 100 cS.

Examples of suitable optional ingredients include:

antioxidants, such as, rosemary extract, tocopherol, a derivative of tocopherol including a tocotriene, carotene, a carotenoid, a phenolic antioxidant including a phenolic acid, a bioflavonoid, a plant extract, curcumin, tetrahydrocurcumin, camphorol, quercetine, epigenine, and any mixtures thereof. The preferred antioxidants are tocopherols and bioflavonoid that have demonstrated antioxidant activity, including ginkgo biloba, pyconogyl pycoogeonyl, pycyogenol, genistein and daidzein, and the like;

keratolytic agents, such as, salicylic acid, resorcinol, peroxide of an organic acid, and any mixtures thereof;

anti-inflammatory agents, such as, steroidal and non-steroidal anti-inflammatory agents and plant extracts that have demonstrated anti-inflammatory activity;

vitamins, such as, Vitamin K, Vitamin C, retinol (vitamin A), tocopherol, and any mixtures thereof;

emollients, such as, cetearyl octanoate, octyl palmitate, butylene glycol, propylene glycol, glycerine, glyceryl monostearate, petrolatum, caprylic trigylceride, capric trigylceride, and shea butter;

humectants, such as, glycerin, propylene glycol, butylene glycol, hyaluronic acid, one or more derivatives of hyaluronic acid, and any mixtures thereof;

skin penetration enhancers, such as, SEPA, butylene glycol, cis-isomer of an unsaturated fatty acid, and any mixtures thereof;

emulsifiers, such as, glyceryl stearate, cetearyl alcohol, cetyl alcohol and PEG-40 stearate;

viscosity control agents, especially thickening agents, such as, xanthan gum and hydroxyethyl cellulose;

retinoids, such as, retinol, one or more esters of retinol, retinoic acid, one or more esters of retinoic acid, a compound that can mimic retinol, and any mixtures thereof;

preservatives, such as, an alkyl paraben, an alcohol, imidazolidinyl urea, and any mixtures thereof;

colorants, such as, synthetic and natural colorants;

chelating agents, such as, disodium EDTA; and

pH adjuster, such as, an acid, a base or a buffer to adjust and maintain the pH to about 6.5 to about 7.5.

Suitable compounds for inclusion in the compositions of the present invention are identified in the International Cosmetic Ingredients Dictionary and Handbook, 9th Edition, 2002, Section 4, incorporated herein by reference.

Preferably, the composition is in the form of a crème or a lotion, but could also be a gel, ointment, mousse, stick, pomade, or spray.

The product can function as a sunscreen, insect repellent, or a combination of a sunscreen and insect repellent, skin care product, hair care, nail care, or lip care or lip color product, a make-up or color cosmetic; a shave crème, a moisturizer, exfoliating product, treatment product, anti acne product, skin protectant product, skin lightening product, anti-aging product, fragrance product; a skin or hair cleansing product, or hair styling or conditioning product, such as, shampoo, hair conditioner, styling mousse, hair treatment preparation, hair coloring product, semi-perm product, oxidation dye, body wash, liquid soap, skin care preparation, lipstick, mascara, color cosmetic, nail care preparation, cream, foam, gel, lotion, solution, pomade, mousse, stick, pump spray, aerosol spray, or any combinations thereof. When formulated as a sunscreen product, the composition is preferably a crème, lotion, pomade, or spray.

### Methods of Use

The present invention provides a method protecting skin from exposure to the sun. The method includes the step of applying topically onto the skin an effective amount of a composition having a sunscreen according to the present invention.

The present invention further provides a method of repelling insects from skin. The method includes the step of applying topically onto the skin an effective amount of a composition having an insect repellent according to the present invention.

The present invention still further provides a method of simultaneously protecting skin from exposure to the sun and repelling insects from the skin. This method includes the step of applying topically onto the skin an effective amount of a composition having both a sunscreen and an insect repellent according to the present invention.

The invention is further described in the following examples, which is intended to be illustrative and not limiting.

Example 1

| | EXAMPLE 1 |
|---|---|
| FORMULATION | Amounts(%) |
| 1. Octyl methoxy cinnamate | 7.5 |
| 2. Oxybenzone | 3.0 |
| 3. Butyl methoxydibenzoylmethane | 2.0 |
| 4. BHT^{b} | 0.1 |
| 5. Trimethylsiloxysilicate/dimethicone^{a} | 67.4 |
| 6. di-trimethylolpropane tetraisostearate | 20.0 |

^{a}dow corning 593 fluid
^{b}Butylated hydroxytoluene

The above formula achieves an SPF of approximately 30 when tested in vivo, according to the method specified in the FDA monograph (FDA sunscreen final monograph as recorded in federal register vol. 64, no. 98, Friday May 21, 1999 pages 27666-27693). Those skilled in the art will appreciate that the level of sunscreen utilized would be expected to yield an SPF of only about 15-20 when incorporated in a typical o/w emulsion.

The performance of the formulations described below as EXAMPLE 2, a formulation according to the present invention, and EXAMPLE 3, a comparative example, further illustrate this point.

### Examples 2 and 3

| | | EXAMPLE 2 | EXAMPLE 3 |
|---|---|---|---|
| FORMULATION | | Amounts(%) | Amounts(%) |
| 1. | Demineralized water | 49.16 | 49.15 |
| 2. | Octyl methoxy cinnamate | 7.5 | 7.5 |
| 3. | Oxybenzone | 3.0 | 3.0 |
| 4. | Butylmethoxydibenzoylmethane | 2.0 | 2.0 |
| 5. | Trimethylsiloxysilicate/ | 27.25 | 25.0 |
| | dimethicone* | | |
| 6. | Benzyl alcohol | 0.99 | 1.0 |
| 7. | Methyl paraben | 0.3 | 0.4 |
| 8. | Propylparaben | 0 | 0.1 |
| 9. | Peg 5 di trimethylolpropane | 0 | 10 |
| | Tetraisostearate | | |
| 10. | Peg 4 lauryl ether | 0 | 0.25 |
| 11. | Peg 100 stearate | 0 | 0.75 |
| 12. | Triethanolamine | 0 | 0.35 |
| 13. | Carbopol 940 | 0 | 0.5 |
| 14. | BHT | 0.1 | 0 |
| 15. | Cyclomethicone pentamer/ | 7.5 | 0 |
| | dimethicone copolyol 90/10** | | |
| 16. | Disodium EDTA | 0.20 | 0 |
| 17. | Sodium Chloride | 2.0 | 0 |

*Dow Corning 593 Fluid
**Dow Corning 5225 Fluid

### Procedure for Example 2

Ingredients 2-4, 6-8, and 14 were added together and heated to about 50° C. with slow agitation until they dissolved and, with gentle prop mixing were allowed to cool back to room temperature. After cooling to room temperature, they produced a single uniform phase, which was the oil phase.

Ingredients 5 and 15 were combined in a beaker at room temperature with gentle prop mixing for about 10 minutes to form a uniform silicone phase. The oil phase was slowly added to this uniform silicone phase with moderate prop mixing such that a vortex was formed without much splashing or incorporation of air, thereby forming an oil in silicone emulsion. This order of addition is very important to ensure that the oil phase is the internal phase.

Mixing was continued for an additional 10 minutes and then ingredients 1, 16 and 17 that had been premixed at room temperature (medium prop mixing for about 15 minutes) until dissolved were slowly added to the oil in silicone emulsion over about 20 minutes with moderate mixing on a prop to form a water phase within the oil in silicone emulsion.

An appropriate mixing speed is one where the vortex formed will absorb the water with a second or two after addition, but where the mixing is not so vigorous as to give splashing.

During this time the emulsion may thicken, so mixing speed may have to be adjusted to maintain a proper vortex. Mixing is continued for an additional 20-30 minutes at the same speed to produce an oil in silicone/water in silicone dual emulsion, after which the emulsion was then poured into jars.

### Procedure for Example 3

Ingredients 1 and 13 were combined with high shear prop mixing to produce a carbopol dispersion, which is the aqueous phase.

Ingredients 2-4, 6-11, and 14 were heated to about 50° C. to melt and combine them into a uniform phase. The uniform phase is then added to ingredient 5 to form an oil in silicone emulsion. This step is carried out at above room temperature because the emulsifier (ingredient 11) is not soluble at room temperature.

The oil in silicone emulsion is then added to the aqueous phase (the carbopol dispersion) with moderate prop mixing, mixed for 10 minutes, and then the triethanolamine is added (ingredient 12) to gel the carbopol.

The mixing is continued for an additional 5 minutes and then poured into jars.

Example 2 formulation has the silicone as the external phase and both the oil and the water as two distinct internal phases. Thus, it is an oil in silicone/water in silicone dual emulsion, which exhibits an SPF of approximately 30. In contrast, Example 3, which has water as the external phase and the oil in silicone emulsion as the internal phase, exhibits an SPF of only 20.

When water is present, water-soluble sunscreens may be dissolved in the water, as illustrated by Example 4.

### Example 4

| | EXAMPLE 4 |
|---|---|
| FORMULATION | Amounts(%) |
| Demineralized water | 47.01 |
| Phenylbenzimidazole sulfonic acid | 1.7 |
| Sodium hydroxide (50% aq) | 0.45 |
| Octyl methoxy cinnamate | 7.5 |
| Oxybenzone | 3.0 |
| Butylmethoxydibenzoylmethane | 2.0 |
| Trimethylsiloxysilicate/dimethicone* | 27.25 |
| Benzyl alcohol | 0.99 |
| Methyl paraben | 0.3 |
| BHT | 0.1 |
| Cyclomethicone pentamer/dimethicone | 7.5 |
| copolyol 90/10** | |
| Disodium EDTA | 0.20 |
| Sodium Chloride | 2.0 |

*Dow Corning 593 Fluid
**Dow Corning 5225 Fluid

The present invention works well with other sunscreens, particularly octocrylene. It is also compatable with inorganic sunscreens and formulations containing inorganic sunscreens, such as, titanium dioxide and zinc oxide.

The silicone resin can be utilized as a powdered resin or as a solution, such as, Dow Corning 593 Fluid, which is a solution of silicone resin in dimethicone.

It should be understood that the foregoing description is only illustrative of the present invention. Various alternatives and modifications thereof can be devised by those skilled in the art without departing from the present invention. Accordingly, the present invention is intended to embrace all such alternatives, modifications and variations that fall within the scope of the appended claims.

What is claimed is:

1. A two phase oil in silicone emulsion composition, adapted to provide an SPF of at least 30, comprising:
from about 1 to about 50 wt % of an internal oil phase that is immiscible with a silicone fluid in the absence of a silicone resin selected from the group consisting of MQ resins, MT resins, MTQ resins, MDQ resins, and MDTQ resins, said oil phase comprising a physiologically acceptable lipophilic and/or hydrophobic sunscreen active ingredient having an octanol-water partition coefficient whose Log P is at least 3.5;
from about 10 to about 70 wt % of a silicone external phase comprising a silicone fluid having a viscosity of up to about 25,000 cS and from 1 to 100 siloxy units; and
a silicone resin selected from the group consisting of MQ resins, MT resins, MTQ resins, MDQ resins, and MDTQ resins in an amount sufficient to emulsify the oil in the silicone fluid to produce the oil in silicone emulsion.

2. The oil in silicone emulsion of claim 1, wherein the lipophilic and/or hydrophobic sunscreen active is selected from the group consisting of oxybenzone, dioxybenzone, menthyl anthranilate, benzophone-3, octyl methoxycinnamate, octocrylene, drometrizole trisiloxane, octyl salicylate, homomethyl salicylate, 4-methyl benzylidene camphor, 3-benzylidene camphor, benzylidene camphor sulfonic acid ester, octyl triazone, phenyl benzimidazole sulfonic acid ester, terephthalydiene dicamphor sulfonic acid ester, di-t-butyl hydroxybenzylidene camphor, dibenzoylmethane derivative, butylmethoxy dibenzoylmethane, terephthalydiene methylene bis-benzotriazolyl-tetramethylbutylphenol, diethylhexyl-2,6-naphthalate, bis-ethylhexyloxyphenol methoxyphenol triazine, benzotriazole derivatives, hydroxy methylphenyl benzotriazole, methylene bis-benzotriazolyltetramethylbutyl phenol, bis-ethylhexyloxyphenol methoxyphenol triazine, hydroxybenzophenone, an oxanilide, cinnamate derivative, a silicone-anchored derivative, and any combinations thereof.

3. The oil in silicone emulsion of claim 2, wherein the sunscreen is present in an amount of from about 10 to about 35 wt % based on the total weight of the emulsion.

4. The oil in silicone emulsion of claim 2, wherein the emulsion exhibits an SPF about 30 to about 100.

5. The oil in silicone emulsion of claim 1, wherein the oil is a branched chain ester formed from an alcohol of 1-5 carbon atoms and a carboxylic acid of 2-28 carbon atoms.

6. The oil in silicone emulsion of claim 1, wherein the oil phase comprises an ester selected from the group consisting of a triester of trimethylolpropane, ditrimethylolpropane tetraisostearate, diisostearyl fumarate, 2-phenylethyl ester of benzoic acid, and any mixtures thereof.

7. The oil in silicone emulsion of claim 6, wherein the carboxylic acid is selected from the group consisting of an acid of 8-22 carbon atoms.

8. The oil in silicone emulsion of claim 7, wherein the oil is an ester selected from the group consisting of a mono-, di-, tri- or tetra-ester of a fatty acid of 12-22 carbon atoms, and any mixtures thereof.

9. The oil in silicone emulsion of claim 8, wherein the ester is selected from the group consisting of a triester of trimethylolpropane, an alkoxylated derivative thereof, a tetraester of di trimethylolpropane, an alkoxylated derivative thereof, and any mixtures thereof.

10. The oil in silicone emulsion of claim 9, wherein the ester is selected from the group consisting of di-trimethylolpropane tetra-isostearate and ethoxy-di-trimethylolpropane-tetra-isostearate.

11. The oil in silicone emulsion of claim 1, wherein the oil is present at about 5 wt % to about 30 wt % based on the total weight of the emulsion.

12. The composition of claim 1, wherein the oil has at least two carbon rings and/or at least one C8 to C22 branched chain.

13. The oil in silicone emulsion of claim 1, wherein the silicone fluid has a viscosity of up to about 1000 cS at 25° C. and is present at about 10 wt % to about 50 wt % of the total weight of the emulsion.

14. The oil in silicone emulsion of claim 1, wherein the silicone fluid is selected from the group consisting of a non-volatile silicone fluid, and a mixture of a non-volatile silicone fluid and a volatile silicone fluid, wherein the volatile silicone fluid is in an amount that is compatible with the stability of the emulsion.

15. The oil in silicone emulsion of claim 14, wherein the non-volatile silicone fluid is dimethicone and the volatile silicone fluid is cyclomethicone.

16. The oil in silicone emulsion of claim 15, wherein the volatile silicone fluid is up to 50 wt % of the total weight of the silicone fluid.

17. The oil in silicone emulsion of claim 1, wherein the silicone resin is about 2.5 wt % to about 30 wt % of the total weight of the emulsion.

18. A two phase oil in silicone emulsion sunscreen composition, comprising:
from about 1 to about 35 wt % of an internal oil phase that is immiscible with a silicone fluid in the absence of an MQ silicone resin, said oil phase having a physiologically acceptable lipophilic and/or hydrophobic sunscreen active ingredient having an octanol-water partition coefficient whose Log P is at least 3.5;
from about 10 to about 50 wt % of a silicone external phase comprising a nonvolatile silicone fluid having a viscosity of up to about 25,000 cS and from 1 to 100 siloxy units;
an MQ silicone resin in an amount sufficient to emulsify the oil in the silicone fluid to produce the oil in silicone emulsion; and
an ingredient selected from the group consisting of antioxidant, buffering agent, botanical extract, chelating agent, pigment, colorant, depigmenting agent, emollient, emulsion stabilizer, film former, fragrance, humectant, lubricant, moisturizer, conditioning agent, preservative, skin protectant, skin penetration enhancer, stabilizer, surfactant, thickener, viscosity modifier, vitamin, and any combinations thereof.

19. The oil in silicone emulsion of claim 18, wherein the MQ resin is trimethylsiloxysilicate present in an amount of up to 30 wt %.

20. The composition of claim 19, wherein the MQ resin is trimethylsiloxysilicate present in an amount of from about 2.5 to about 15 wt %.

21. A method of protecting skin from exposure to the sun, comprising:
applying topically onto the skin an effective amount of a composition according to claim 18.

22. An oil in silicone emulsion, comprising:
an oil phase having an effective amount of a lipophilic and/or hydrophobic active ingredient;
a silicone phase having a silicone fluid; and
a silicone resin in an amount sufficient to produce the oil in silicone emulsion; wherein the silicone resin is selected from the group consisting of MQ resins, MT resins, MTQ resins, MDQ resins, and MDTQ resins.

23. The oil in silicone emulsion of claim 22, wherein the silicone emulsifying resin is an MQ resin.

24. The oil in silicone emulsion of claim 23, wherein the MQ resin is trimethylsiloxysilicate present in an amount of from about 2.5 to about 30 wt %.

25. The oil in silicone emulsion of claim 24, wherein trimethylsiloxysilicate is present in an amount of from about 2.5 to about 30 wt %.

26. The composition of claim 22, wherein the active ingredient is a sunscreen.

27. A two phase waterproof oil in silicone emulsion composition, adapted to provide an SPF of at least 30, comprising:
from about 1 to 35 wt % of an internal oil phase comprising an oil selected from the group consisting of a branch chain ester formed from an alcohol selected from the group consisting of ditrimethylolpropane, pentaerythritol, and mixtures thereof, and a carboxylic acid of 2-28 carbon atoms; an ester selected from the group consisting of a triester of trimethylolpropane, an alkoxylated derivative thereof, a tetraester of ditrimethylolpropane, an alkoxylated derivative thereof, and mixtures thereof, said oil phase having a physiologically active lipophilic and/or hydrophobic sunscreen active ingredient;
from about 10 wt % to about 50 wt % of a silicone external phase comprising a nonvolatile silicone fluid having a viscosity of up to about 25,000 cS and from 1 to 100 siloxy units; and
a silicone resin selected from the group consisting of MQ resins, MT resins, MTQ resins, MDQ resins, and MDTQ resins, in an amount sufficient to emulsify the oil in the silicone fluid to produce the oil in silicone emulsion.

28. The oil in silicone emulsion of claim 27, wherein the MDTQ resin is an MQ resin present in an amount of up to 30 wt %.

29. The oil in silicone emulsion of claim 28, wherein the MQ resin is trialkylsiloxysilicate.

30. A process for preparation of a waterproof two phase oil in silicone emulsion, comprising the steps of:
mixing one or more cosmetic active ingredients and optionally an oil and/or an adjuvant at shear at a temperature and for a period of time sufficient to produce an oil phase having an effective amount of a lipophilic and/or hydrophobic active ingredient;
mixing a silicone fluid and a silicone resin selected from the group consisting of MQ resins, MT resins, MTQ resins, MDQ resins, and MDTQ resins, and optionally an adjuvant at shear at a temperature and for a period of time sufficient to produce a silicone phase; and
adding the oil phase to the silicone phase at shear at a temperature and for a period of time sufficient to produce the oil in silicone emulsion.

31. The process of claim 30, wherein the MDTQ resin is an MQ resin present in an amount of up to 30 wt %.

32. The process of claim 31, wherein the MQ resin is trialkylsiloxysilicate.
